(19) United States
(12) Patent Application Publication
(10) Pub. No.: US 2006/0280696 A1
(43) Pub. Date: Dec. 14, 2006

(54) Method For Treating Periodontal Disease

(75) Inventor: Steven D. Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO Group, Inc., Sandy, UT (US)

(21) Appl. No.: 11/382,586

(22) Filed: May 10, 2006

Related U.S. Application Data
(60) Provisional application No. 60/689,365, filed on Jun. 10, 2005.

(51) Int. Cl. A61K 8/22; A61K 8/19; A61K 8/43; A61C 5/00

(52) U.S. Cl. 424/49; 433/217.1; 424/53

(57) ABSTRACT

Present invention provides a method for treating periodontal disease. Periodontal disease is a pathogenic infection of the gums. The method comprises the use of a laser or radiant energy source that is capable of being absorbed by pathogens. Said Radiant energy is applied to infected periodontal pockets with the intention of destroying any susceptible pathogens. The periodontal pocket is then flushed with an anti-microbial substance with the intention to destroy any residual susceptible pathogens.

### CROSS REFERENCE TO RELATED APPLICATIONS

This application claims priority as a non-provisional perfection of prior filed provisional application 60/689,365, filed Jun. 10, 2005.

### FIELD OF THE INVENTION

The present invention relates to the field of treatment of periodontal disease and more particularly relates to treating periodontal disease with a laser and chemical combination.

### BACKGROUND OF INVENTION

Periodontal disease is a pathogenic infection of the gums and it is common among all humans and animals. Periodontal disease is a major cause in the loss of teeth and oral bone throughout every society. The oral environment is a warm moist cavity that is full of nutrients; it is an excellent location to incubate numerous microbes. Therefore it is not surprising that pathogens readily ingress into the periodontal pockets and begin causing infection. Uncontrolled or rampant periodontal infection leads to bone loss that ultimately results in the teeth becoming loose from their sockets.

Until now, treatments have involved extensive and painful processes to clean the infected area and the use of drugs to control the infection. Recently, lasers have been used to treat periodontal disease by using a fiber-optic guide to direct laser energy into periodontal pockets to kill bacteria. This less invasive and painful form of treatment does have its limitations, however, in that the laser is limited by the relative size of the guide and the ability to adequately control its direction. As such, areas needing treatment may not be adequately treated or missed entirely. What is needed is a method to improve upon the use of the laser treatment of periodontal disease for maximum coverage and disinfection of the treated area.

### BRIEF SUMMARY OF THE INVENTION

In view of the foregoing disadvantages inherent in the known types of treatment of periodontal disease, this invention provides a new and improved method of treatment merging the benefits of laser and chemical treatment. As such, the present invention's general purpose is to provide a new and improved method that is safe, efficient, with minimal discomfort to the patient and providing a broader treatment area than the use of the laser guide alone.

The present invention provides an improved method for treating periodontal disease. The method comprises the use of a laser or radiant energy source that is capable of being absorbed by pathogens. Said laser light is applied to infected periodontal pockets with the intention of destroying any susceptible pathogens. The periodontal pocket is then flushed with an anti-microbial substance with the intention to destroy any residual susceptible pathogens. The advantage of the flushing is that any residual organisms have been already weakened by the applied laser light and the use of a liquid anti-microbial substance will reach areas missed by the direction of the guide.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a partial sectional view of a normal tooth and surrounding tissue.

FIG. 2 is the tooth and surrounding tissue of FIG. 1 having developed an early stage of gingivitis.

FIG. 3 is the tooth and surrounding tissue of FIG. 2 having developed advanced periodontal disease.

FIG. 4 is the tooth and surrounding tissue of FIG. 3, being treated by a fiber optic guide through which laser light is being transmitted.

FIG. 5 is tooth and surrounding tissue of FIG. 4 being flushed with an anti-microbial substance by the means of a slender tip attached to a syringe.

### DETAILED DESCRIPTION OF THE INVENTION

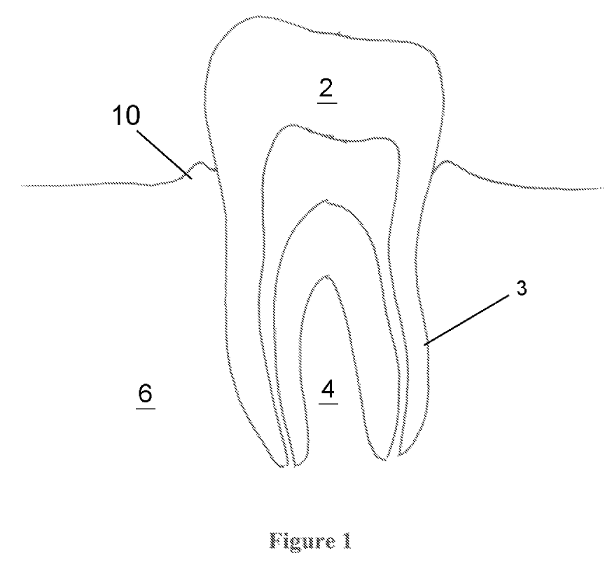

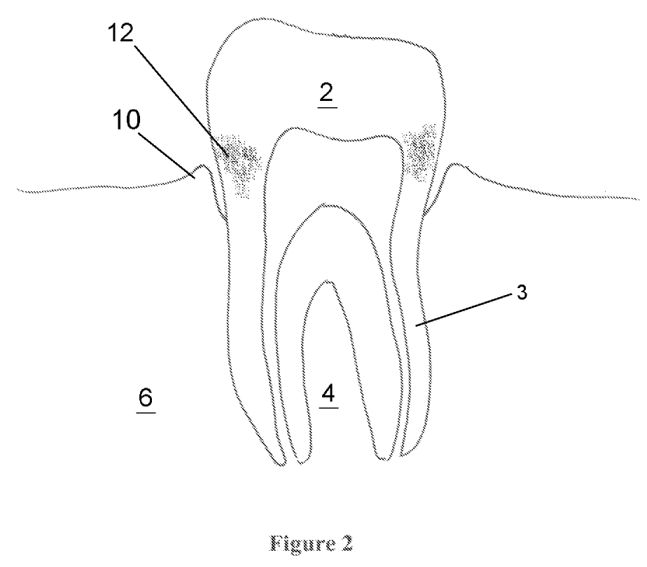

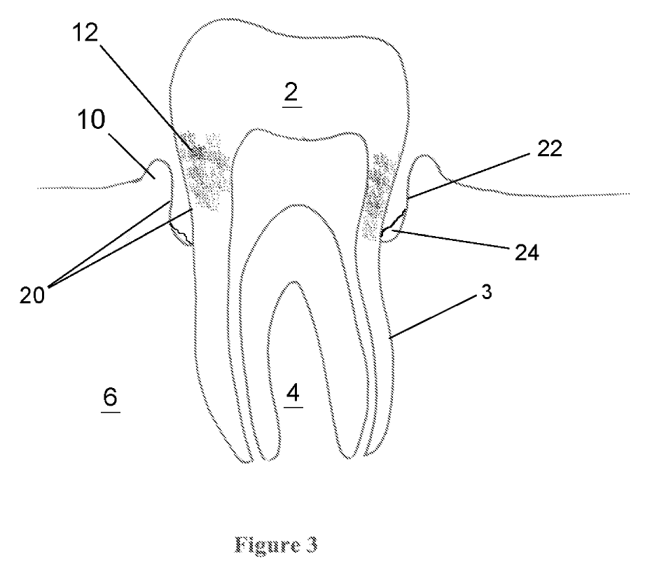

With reference now to the drawings, the preferred embodiment of the method of periodontal treatment is herein described. It should be noted that the articles “a”, “an” and “the”, as used in this specification, include plural referents unless the content clearly dictates otherwise. With reference to FIG. 1, a healthy tooth 2 rests in a bony socket 4 in the jaw 6. The entire area is covered by the gingiva 10, or “gums”, to protect the juncture. Over time, if left without proper oral care, tartar 12 will build up against tooth 2 (shown in FIG. 2), causing the gums 10 to recede and exposing the root 3 of the tooth in a condition called “gingivitis”. FIG. 3 displays a condition further deteriorated from gingivitis, peridontitis. The gums 10 have receded to the point of forming an open pocket 20 around the tooth 2 and its root system 3. The pocket is filled with inflamed tissue 22 and infectious matter 24. If left untreated the tooth 2 and socket 4 may deteriorate, causing loss of the tooth 2.

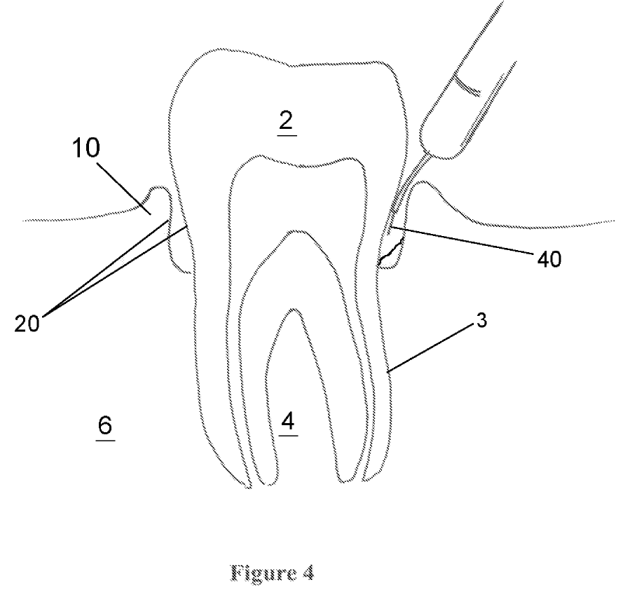

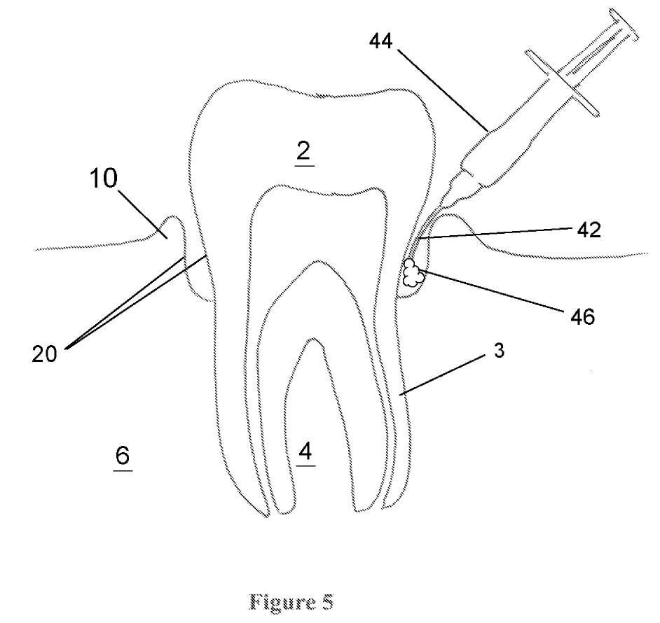

Treatment of the condition is shown in FIGS. 4 and 5. The method harnesses the benefits of a radiant energy source that is lethal to pathogens, coupled with anti-microbial agents that are chemically lethal to a wide variety of pathogens. The combined effect of radiant energy bombardment and the additional challenge of anti-microbial agents is intended to destroy a broad spectrum of pathogens; such that remaining pathogens can eventually be controlled by the normal functions of the immune system.

The method warrants a radiant energy source with sufficient energy to become lethal to pathogens. The radiant energy can be produced from sources such as a diode laser, examples of which are the gallium nitride, aluminum gallium arsenide diode laser and the like. The radiant energy can be produced from sources such as high intensity light from incandescent, halogen or plasma arc devices. The radiant energy can be produced from sources such as solid state lasers, examples of which are neodymium YAG, titanium sapphire, thulium YAG, ytterbium YAG, Ruby, holmium YAG lasers and the like. The radiant energy can be produced from sources such as EB or electron beam devices. The radiant energy can be produced from sources such as gas lasers, examples of which are the Carbon dioxide laser, argon gas, xenon gas, nitrogen gas, helium-neon gas, carbon monoxide gas, hydrogen fluoride gas lasers and the like. There are also many dye lasers that utilize a radiant energy source that pass through various dyes or stains to achieve various wavelengths. Dye lasers are also within the scope of this method.

The method also warrants an anti-microbial substance that is capable of destroying pathogens. There are numerous substances with anti-microbial or anti-pathogenic activity. Any substance that is capable of destroying or stemming the growth of a pathogen is within the scope of this method. A few possible examples of antimicrobial substances include: ethanol, isopropanol, methyl paraben, ethyl paraben, butyl paraben, propyl paraben, hydrogen peroxide, carbamide peroxide, eugenol, sodium chlorite, chlorhexidine, chlorhexidine gluconate, sodium chlorite, thymol, cetyl pyridinium chloride, chloroxylenol, iodine, hexachlorophene, triclosan, quaternary ammonium compounds, sodium hypochlorite, calcium hypochlorite, or any like substance that is capable of destroying or limiting the reproduction of pathogens.

Many of these antimicrobial agents are a dry powder in their raw form and would benefit by being dissolved into a solvent. Liquid antimicrobial agents are able to migrate easier into difficult areas, thus having an advantage over powders. A few examples of possible solvents include: water, propylene glycol, glycerin, polysorbates, liquid polyethylene glycols, ethanol or any solvent capable of dissolving or liquefying an antimicrobial substance.

Optionally, the antimicrobial agent can contain additional components that would improve patient comfort such as a flavor, sweetener or anesthetic. A few possible substances that would aid in patient comfort include: sodium saccharin, phenylalanine, benzocaine, lidocaine, dyclonine hydrochloride, peppermint oil, spearmint oil, methyl salicylate and any like substance.

Numerous formulas are capable of being produced during the practice of this method. Compositions may be made in any combination according to the following Table A, dependant upon the desired agents used and overall effect.

TABLE A

| Rinse | Percentage by | |
|---|---|---|
| component | total weight | Function |
| Antimicrobial | 0.01%%-100% % | Kill bacteria |
| agent | | |
| Solvent | 0%-99.99% | Allows the rinse |
| | | to be a fluid that |
| | | will easily flow |
| | | into a periodontal |
| | | pocket. |
| Flavoring | 0%-5% | Make the rinse |
| | | palatable. |
| Anesthetic | 0%-30 | Reduce patient |
| | | discomfort. |

A few specific examples could include:
Formula #1
6.0%—chlorhexidine gluconate 20% aqueous
94.0%—Water
   Formula #2
1%—chlorhexidine
99.0%—Water
   Formula #3
5.0%—sodium hypochlorite
95.0%—Water
   Formula #4
1.0%—calcium chlorite
99.0%—Water
   Formula #5
0.5%—sodium chlorite
99.5.0%—Water
   Formula #6
10.0%—chlorhexidine gluconate 20% aqueous
73.4%—Water
0.3%—peppermint oil
15.0%—ethanol
0.3%—Phenylalanine
1.0%—dyclonine hydrochloride
   Formula #7
3.0%—hydrogen peroxide
55.4%—glycerin
0.3%—peppermint oil
40.0%—water
0.3%—Phenylalanine
1.0%—benzocaine
   Formula #6
1.0%—methyl paraben
25.0%—Water
0.3%—methyl salicylate
25.0%—ethanol
0.3%—sodium saccharin
1.0%—lidocaine
47.4%—propylene glycol
   The above example formulas would be sufficiently adequate over one or multiple applications to destroy or limit the growth of pathogens in the oral environment.

A typical procedure of events during a routine periodontal treatment regime would be to first identify areas of greatest infection. These areas would be selected for additional exposure to radiant energy. The radiant energy source would then be focused into these infected pockets by means of a thin fiber optic guide 40, FIG. 4. The fiber optic guide being small enough to be directed between the teeth and gums. The periodontal pocket 20 is then radiated with radiant energy while the optical fiber 40 is moved in increments around the gums 10. When the treatment of the gums by radiant energy is complete, the periodontal pocket 20 is then flushed with an antimicrobial fluid 46 by means of a small tip 42 attached to a syringe 44, shown in FIG. 5. The treatment regime may include multiple treatments, these factors depend on the degree of infection present. The treatment regime usually continues until the swelling and redness of infected gums is no longer apparent and only pink healthy gums persist.

The treatment regime can also begin by flushing the periodontal pockets with antimicrobial agents, followed by radiating with radiant energy. This would allow any additional anisthetic contained in the antimicrobial agent to anesthetize the working area prior to receiving radiant energy.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method of treating periodontal disease comprising:
a. An initial step of directly applying lethal radiant energy to susceptible oral pathogens in infected areas, and
b. A secondary step of flushing radiated areas with a fluid containing an antimicrobial agent.

2. The method of claim 1 further comprising the step of directing the radiant energy to the infected areas with a flexible fiber optic guide.

3. The method of claim 2, further comprising the step of generating the radiant energy from a source selected from the set of sources consisting of: a gas laser, a solid state laser, a diode laser, and an 810 nm diode laser.

4. The method of claim 3, the antimicrobial agent being selected from the set of antimicrobial agents consisting of: chlorhexidine gluconate, chlorhexidine, hydrogen peroxide, sodium hypochlorite, and sodium chlorite.

5. The method of claim 2, the antimicrobial agent being selected from the set of antimicrobial agents consisting of: chlorhexidine gluconate, chlorhexidine, hydrogen peroxide, sodium hypochlorite, and sodium chlorite.

6. The method of claim 1, further comprising the step of generating the radiant energy from a source selected from the set of sources consisting of: a gas laser, a solid state laser, a diode laser, and an 810 nm diode laser.

7. The method of claim 6, the antimicrobial agent being selected from the set of antimicrobial agents consisting of: chlorhexidine gluconate, chlorhexidine, hydrogen peroxide, sodium hypochlorite, and sodium chlorite.

8. The method of claim 1, the antimicrobial agent being selected from the set of antimicrobial agents consisting of: chlorhexidine gluconate, chlorhexidine, hydrogen peroxide, sodium hypochlorite, and sodium chlorite.

9. A method of treating periodontal disease comprising:
a. An initial step of directly flushing infected areas containing susceptible oral pathogens with a fluid containing an antimicrobial agent, and
b. A second step of applying lethal radiant energy to previously flushed areas.

10. The method of claim 9, further comprising a third step of directly flushing previously flushed and treated areas with a fluid containing an antimicrobial agent.

11. The method of claim 10 further comprising the step of directing the radiant energy to the infected areas with a flexible fiber optic guide.

12. The method of claim 11, further comprising the step of generating the radiant energy from a source selected from the set of sources consisting of: a gas laser, a solid state laser, a diode laser, and an 810 nm diode laser.

13. The method of claim 12, the antimicrobial agent being selected from the set of antimicrobial agents consisting of: chlorhexidine gluconate, chlorhexidine, hydrogen peroxide, sodium hypochlorite, and sodium chlorite.

14. The method of claim 11, the antimicrobial agent being selected from the set of antimicrobial agents consisting of: chlorhexidine gluconate, chlorhexidine, hydrogen peroxide, sodium hypochlorite, and sodium chlorite.

15. The method of claim 10, further comprising the step of generating the radiant energy from a source selected from the set of sources consisting of: a gas laser, a solid state laser, a diode laser, and an 810 nm diode laser.

16. The method of claim 15, the antimicrobial agent being selected from the set of antimicrobial agents consisting of: chlorhexidine gluconate, chlorhexidine, hydrogen peroxide, sodium hypochlorite, and sodium chlorite.

17. The method of claim 10, the antimicrobial agent being selected from the set of antimicrobial agents consisting of: chlorhexidine gluconate, chlorhexidine, hydrogen peroxide, sodium hypochlorite, and sodium chlorite.

18. The method of claim 9 further comprising the step of directing the radiant energy to the infected areas with a flexible fiber optic guide.

19. The method of claim 18, further comprising the step of generating the radiant energy from a source selected from the set of sources consisting of: a gas laser, a solid state laser, a diode laser, and an 810 nm diode laser.

20. The method of claim 19, the antimicrobial agent being selected from the set of antimicrobial agents consisting of: chlorhexidine gluconate, chlorhexidine, hydrogen peroxide, sodium hypochlorite, and sodium chlorite.

21. The method of claim 18, the antimicrobial agent being selected from the set of antimicrobial agents consisting of: chlorhexidine gluconate, chlorhexidine, hydrogen peroxide, sodium hypochlorite, and sodium chlorite.

22. The method of claim 9, further comprising the step of generating the radiant energy from a source selected from the set of sources consisting of: a gas laser, a solid state laser, a diode laser, and an 810 nm diode laser.

23. The method of claim 22, the antimicrobial agent being selected from the set of antimicrobial agents consisting of: chlorhexidine gluconate, chlorhexidine, hydrogen peroxide, sodium hypochlorite, and sodium chlorite.

24. The method of claim 9, the antimicrobial agent being selected from the set of antimicrobial agents consisting of: chlorhexidine gluconate, chlorhexidine, hydrogen peroxide, sodium hypochlorite, and sodium chlorite.
